(19) United States
(12) Patent Application Publication
(10) Pub. No.: US 2018/0014871 A1
(43) Pub. Date: Jan. 18, 2018

(54) Internal Tissue Heating Apparatus

(71) Applicants: Hirak Mitra, Santa Clara, CA (US); Donald Carson Lewis, Richmond, CA (US)

(72) Inventors: Hirak Mitra, Santa Clara, CA (US); Donald Carson Lewis, Richmond, CA (US)

(21) Appl. No.: 15/207,792

(22) Filed: Jul. 12, 2016

(51) Int. Cl. A61B 18/12; A61M 37/00; A61N 1/40; A61B 18/00

(52) U.S. Cl. CPC A61B 18/1206; A61N 1/403; A61M 37/00; A61B 2018/00577; A61B 2018/1273; A61M 2037/0007

(57) ABSTRACT

A machine holds a plurality of RF beam generators which all point at a particular location. A patient with a target volume of tissue to be treated by heat is held immobile such that the target is located at the point all the RF beams traverse. The RF beams traverse that point and the resultant generated heat treats the target. No two or more RF beams traverse any other location in the patient's body. Since each individual RF beam is too weak to cause damage, the non-targeted tissues of the patient are unharmed.

### REFERENCES

1. Radiofrequency Ablation of Stage IA Non-Small Cell Lung Cancer in Medically Inoperable Patients: Results From the American College of Surgeons Oncology Group Z4033 (Alliance) Trial, Dupuy et al.
2. https://en.wikipedia.org/wiki/Maser
3. http://www.virtualtrials.com/gamma.cfm
4. https://en.wikipedia.org/wiki/Microwave
5. http://www.kurzweilai.net/new-metamaterial-lens-focuses-radio-waves
6. http://www.cancer.org/treatment/treatmentsandsideeffects/treatmenttypes/hyperthermia
7. https://en.wikipedia.org/wiki/John Kanzius#Cancer_therapy
8. http://www.nature.com/news/2008/081017/full/news.2008.1178.html
9. http://www.ncbi.nlm.nih.gov/pubmed120729423
10. https://en.wikipedia.org/wiki/Electromagnetic absorption_by_water
11. http://cancer.ucsd.edu/treatments/heated-intraperitoneal-chemotherapy/Pages/how-HIPEC-works.aspx
12. http://www.ncbi.nlm.nih.gov/pmc/articles/PMC3323111/

### PRIORITY

This disclosure claims the priority, and includes the contents, of US PPA 62/025,496, filed on Jul. 17, 2014.

### FIELD

This pertains to the field of hyperthemia, heating particular internal tissue in vivo, without heating the tissue around it. A mechanism to do this will pertain to various methods in the field of medicine, including but not limited to:
oncology and in particular tumor ablation by heat-induced destruction of tissue
oncology and in particular tumor ablation by heat in conjunction with other techniques
internal cauterization of wounds and defects such as ulcers and fatty deposits

### BACKGROUND

There are a variety of ailments which respond positively to the judicious application of heat. A common and successful approach to ablating a cancerous tumor is to apply heat to it. This works both on its own to destroy tumorous tissue outright; and also in conjunction with other treatments such as some forms of chemotherapy, nanoparticle therapy and irradiation treatment which render tumors more susceptible to destruction by heat.

Another use of heat is to cauterize a wound to prevent it from leaking fluid.

Other uses of heat may include the option to ablate other tissue, such as intestinal ulcers, fatty deposits, torn cartilage, or a swollen appendix or prostate.

The deleterious side effect of applying heat is that sufficient heat damages the tissue surrounding the tissue intended to receive the heat, as well as the tissue thus intended, called the target, itself. This is because sufficient heat alone will denature or “cook” cellular components and structures, and ultimately destroy cells and thus living tissue; tumorous, healthy or otherwise. Non-target tissue refers to tissue not intended for treatment.

An interesting aspect of applying heat to tissue is that there is a threshold for heat damage. Applying heat below this threshold causes no damage, while applying heat above this threshold does cause tissue damage.

If the target is at the surface of the body then applying heat to it is trivial, since a course of treatment may apply such heat to the exterior of the body at the site of the target, thereby not directly heating any other tissue in the body. The problem is when the target is not at the surface of the body but rather internal. Then to apply heat to such a target requires heating at least some tissue between the target and some surface of the body.

A way to avoid this is by inserting a heating element into the body and placing this element in or adjacent to said target, causing hyperthermia as shown in [6]. But this requires surgery or some other invasive procedure, and such procedures lead to secondary deleterious side effects such as some level of systemic shock and the formation of scar tissue. Also all invasive techniques carry the risk of introducing infectious cells, toxins and pathogens into the body.

### PRIOR ART

Radiofrequency ablation of tumors is a technique to apply heat directly to a tumor. It is an experimental technique with considerable empirical success, as shown in [1]. The tool used is a probe which is placed into the tissue to be treated by means of surgical incision or catheterization.

A maser is a device that transmits coherent electromagnetic radiation in the infrared and microwave ranges. Masers are well known and well understood in the Art, as seen in [2]. There are now cheap masers as small as a grain of rice. Masers produce well-directed beams of RF energy in the microwave frequency range, as seen in [4]. This range is 300 MHz and 300000 GHz.

RF electromagnetic energy has many frequencies where it is absorbed by liquid water, as shown in [10]. At a frequency where liquid water absorbs RF, that absorption dissipates some of that electromagnetic energy into heat, which raises the temperature of that water. Since tissue comprises mostly water by weight, RF traversing tissue at these frequencies heats the traversed tissue.

Directed radio frequency (RF) devices have also existed for many decades. Such a device may be designed to transmit almost all of its energy in a cylindrical beam, or in a narrow cone where change in the diameter between two parallel cross-sections (frustra) of the beam, is much less than the distance between those two cross-sections. Such devices are well known and well understood in the Art. Metamaterial lenses are capable of focusing RF beams into an arbitrarily small focus as seen in [5]. Together we call masers and directed RF devices, RF beam generators.

Gamma knife radiosurgery [3] is a technique that relies on a machine similar in geometry to the invention disclosed here. A gamma knife comprises a plurality of gamma ray sources. Usually such a source contains a mass of Cobalt₆₀. These sources generate gamma rays. These gamma rays are focused by collimators into narrow beams. These beams are designed geometrically to converge at a small focal point. A patient is placed in such a manner that a tumor is at the focal point. Any individual beam of gamma radiation is weak. But the tumor at the focal point where all the beams converge receives the energy of all the beams simultaneously, and this ablates the tumor. The residual beams of gamma radiation then pass through other tissue any of which receives only a weak dose of radiation and is relatively unscathed. Such gamma knives have existed since the 1960's, and they are well known and well understood in the Art.

A different non-invasive method for heating tissue is focused ultrasound, as shown in [9]. This technique generates a plurality of beams of ultrasound that converge at one focus in the body in a manner similar to the gamma knife. As a beam of ultrasound traverses tissue it dissipates and part of its energy turns to heat. A given beam of ultrasound does not sufficiently heat tissue at any point to cause tissue damage. However at the point these beams converge they dissipate enough heat to ablate tissue. RF treatment may be used in conjunction with this treatment.

RF treatment may be used in conjunction with other forms of therapy such as binding nanoparticles preferentially to cancer cells, as shown in [7].

Heat plus chemotherapy as shown in [11] has been found to be more effective for treating tumors in some cases, than either heat or chemotherapy alone.

Heat plus nanoparticle treatment as shown in [12] have been found efficacious in treating tumors. RF and other tissue heating techniques may be monitored by some means to measure the temperature of the tissue being heated. There are currently non-invasive techniques in development to achieve this, as shown in [8]. In addition invasive techniques exist as well, including needlepoint thermometers. These latter are well known and well understood in the Art.

### SUMMARY OF THE APPARATUS AND METHOD

In an embodiment of the invention: A machine comprises a plurality of radio frequency (RF) beam generators. These RF beam generators are spatially separate but geometrically oriented in such a way that all of the RF beams they generate, pass through a single locality with the quality that the smallest sphere which completely contains this locality has a diameter between 1 and N times the diameter of a single RF beam where N is determined by the geometry of the particular implementation of the machine. This locality is called the focus of the machine.

Living tissue containing a target is placed and held such that the target is placed at the focus. The RF beam generators are energized and thereupon produce RF beams, this process being calling firing. Each RF beam is tuned to a frequency such that as the beam traverses tissue it dissipates in some part of its energy in that tissue, and that dissipated energy converts to heat inside that tissue. Each beam attenuates exponentially as it traverses tissue.

The individual RF beams do not intersect except at the focus. So any given piece of tissue not at the focus has at most one RF beam traversing it. Since each individual RF beam expends little energy traversing tissue, any tissue not at the focus has little RF energy dissipating into it as heat, and this heat energy is too low (beneath the aforementioned threshold) to cause tissue damage. So the tissue not at the focus is not damaged. At the focus where all the RF beams the machine generates intersect, each individual RF beam still expends little energy, but the cumulative energy dissipation is enough to cause damage by exceeding the threshold for thermal damage. This serves to ablate the tissue.

After a given RF beam traverses the focus it continues through other tissue. But at this point the RF beam is already attenuated from prior dissipation. And any such tissue receives at most one RF beam. And the heat energy such an RF beam dissipates in this tissue is insufficient to reach the threshold for thermal damage. So again such tissue is not damaged.

### BRIEF DESCRIPTION OF THE DRAWINGS

FIG. 1 shows an embodiment of the invention. A machine (101) holds RF beam generators (102) in such a way that the RF beams (103) created by energized RF beam generators all pass through a focus (105). The patient (104) is held in the cavity of the machine (106) in such a way that the target is at the focus when the RF beams are energized.

FIG. 2 shows the same embodiment of the invention as FIG. 1. But now instead of the patient there is a calibration sensor (107) that is placed at where the focus is estimated to be. The calibration sensor enables the machine to auto-calibrate, to adjust the geometry and location of its focus, and to determine the exact spatial location of that focus.

### EMBODIMENTS

### Typical Machine

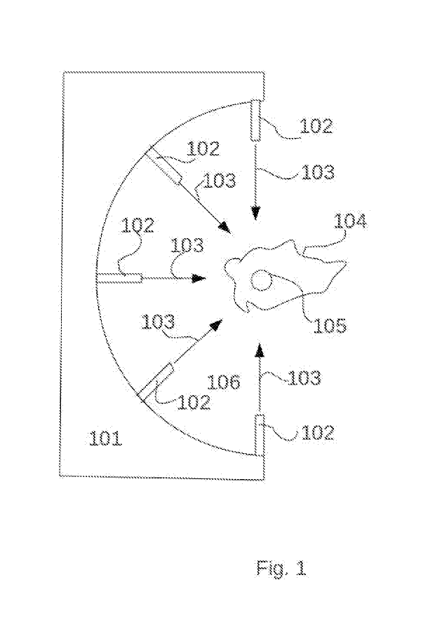

In one typical embodiment in FIG. 1: A machine (101) contains a a surface which fully or partially encloses a cavity (106) 2m across and shaped as a section of a sphere. This surface holds 127 RF beam generators (102) which are geometrically oriented to emit RF beams orthogonally from the surface. The RF beam generators, not all of which are shown, are situated in a hexagonal array where each side of the hexagon comprises 6 RF beam generators. Each RF beam generator sits 20 cm from the RF beam generators nearest to it. Each RF beam generator fires an RF beam 2 mm wide. The RF beam generators are spatially arranged such that when they fire, all of their RF beams (103) pass through a focus (105) which is in the range of [2 mm, 10 cm] in diameter. Each RF is tuned to emit a beam at 1000 GHz frequency.

Note that an RF beam has a resolution of its edges on the order of its wavelength. At 1000 GHz the wavelength is 0.03 cm. So the uncertainty of the edge of the RF beam is around 0.03 cm in this case. A patient (104) is placed in or near the cavity (106) and held immobile such that the target of the patient is held immobile at the focus (105). When the RF beam generators fire any given location in the patent other than the focus has at most one RF beam passing through it. But at the focus all the RF beams intersect.

A computer controls, at a minimum, the operational aspects of the machine, including positioning and tuning the RF array. Personnel program the machine to specify firing patterns for the RF beam generators. These firing patterns determine among other things, which and how many of the RF beam generators fire at a given time, how long the RF beam generators fire, and the intensity of each individual RF beams.

The machine also comprises a non-invasive temperature measurement device which determines the temperatures reached by both the focus tissue, and by the surrounding tissue. This provides feedback to the machine and the machine uses this information to change the intensity and the duration of the beams.

### Typical Method to Calibrate Machine

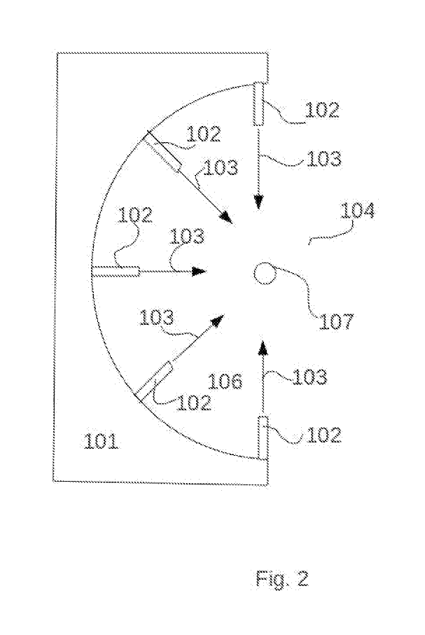

The machine auto-calibrates as follows, as shown in FIG. 2. A sensor (107) is placed at the exact location the personnel operating the machine want the focus to be. Then each RF beam generator (102) calibrates, one at a time, as follows. The machine controls the exact orientation of a RF beam generator by means of servo-motors (not shown) at the base of the generator, where it mounts into the rest of the machine. The machine moves the RF beam generator through its possible positions. At each position the RF beam generator fires an RF beam. When the RF beam intersects the sensor the sensor sends information to the machine that this RF beam generator is properly oriented. When this happens the machine locks this RF beam generator at this position.

One algorithm to do this is summarized as:

```
    place sensor at the desired focus
    for each RF generator rf_gen
    {
        for each possible position pos of rf_gen
        {
        put rf_gen at pos
        fire rf_gen
        if (the generated beam intersects adequately with sensor)
        {
            lock rf_gen at pos
            stop iterating rf_gen through positions
        }
    }
}
```

### Typical Method to Use the Machine

In some embodiments: First the machine auto-calibrates and is cognizant of the exact location of the focus. Then a human being or other living organism, called the patient, has a tumor, internal wound, ulcer, fatty deposit or other tissue medical personnel wish to treat with heat, this tissue being the target. The exact location of the target inside the body of the patient is discovered using various methods; some of these methods are well known and well understood in the Art, while others are the subject of current research and experimentation, and still others are yet to be discovered or invented. All or part of the body of the patient is held immobile inside or near the machine in such a manner that the exact location of the target is at the focus. Some or all of the RF beam generators fire, and the RF beams from these RF beam generators converge at the target. Every individual RF beam carries some amount of power which dissipates as it traverses bodily tissue in the patient and thereby heats that tissue. But any given RF beam is not sufficiently energetic per se to cause tissue damage along its path.

Where the RF beams converge at the focus, where the target is, all of the RF beams dissipate some amount of energy, and again the energy dissipation and resultant heat from any given RF beam is insufficient to cause damage. But the sum of all the energy dissipated by all the RF beams, and the heat therefrom, convergent at the focus, is sufficient to heat the target enough to treat it.

The machine holds a non-invasive temperature-sensing mechanism to measure the temperature at the focus and also in other tissue. This temperature-sensing mechanism provides feedback to the machine regarding how much power to apply through RF energy.

### Alternative Embodiments

In some embodiments RF generators are masers. In some embodiments RF generators are directed RF devices. In some embodiments RF generators are antenna arrays. In some embodiments RF generators are some other RF spectrum energy generating devices. In some embodiments RF generators are some mix of these kinds of devices.

In some embodiments an individual RF beam is focused by a metamaterial lens external to the patient, or some other means, into a smaller convergence than it would have otherwise.

In some embodiments the machine comprises a curved concave surface wherein some number of RF beam generators are embedded such that they point from this curved concave surface. In some further embodiments the curvature of this surface is spherical. In some further embodiments the curvature of this surface is parabolic. In some further embodiments this surface has some other curvature. In some further embodiments the RF beam generators point orthogonally from the curved concave surface, and in some further embodiments the RF beam generators do not.

In some embodiments the machine comprises a flat surface and multiple RF beam generators sit on this surface, each with an angular offset such that all of the RF beams will still pass through a focus.

In some embodiments there is no single surface that holds all the RF beam generators, nor a concavity defined. In such embodiments the machine simply comprises and controls RF beam generators that are oriented such that all their beams meet at a single focus.

In some embodiments some subset, which is potentially the whole set, of the RF beam generators are fixed in position. In some embodiments the positions of some such subset are adjustable by hand labor.

In some embodiments the positions of some such subset are controlled by servomotors and the machine changes the position of each such RF beam generator through its programming.

In some embodiments some subset, which is potentially the whole set, of the RF beam generators sit at fixed orientations in the machine. In some embodiments the orientations some such subset may be adjusted by hand labor. In some embodiments the orientations of some such subset are controlled by servomotors and the machine changes the orientation of each such RF beam generator in the subset through its programming. In some further embodiments the machine calibrates the RF beam generators of the subset by the algorithm presented here. In some other further embodiments the machine, potentially in concert with the operators of the machine, calibrates the RF beam generator by some other algorithm.

In some embodiments the machine holds 127 RF beam generators. In some embodiments the machine holds some other number of RF beam generators.

In some embodiments some subset, which is potentially the whole set, of the RF beam generators sit in a hexagonal array. In some embodiments some such subset sit in some other geometric arrangement. In some embodiments some such subset follows no specific geometric pattern.

In some embodiments the RF beam generators are 20 cm apart. In some embodiments the RF beam generators are some other distance apart.

In some embodiments the RF beam generators emit beams at 1000 GHz. In some embodiments the RF beam generators emit beams at some other frequency. In some embodiments some RF generators are tunable to emit RF at different frequencies. In some embodiments different RF generators are tuned to emit RF at different frequencies at the same time.

In some embodiments the RF beams are 2 mm wide. In some embodiments the RF beams have some other diameter. In some embodiments different RF beams have different diameters.

In some embodiments an RF beam is cylindrical. In some embodiments an RF beam is conical. In some some other embodiments an RF beam is some other shape. In some embodiments different RF beams have differing shapes drawn from this set.

In some embodiments the focus is 2 mm wide. In some embodiments the focus has some other diameter, between 0.001 mm and 10 cm.

In some embodiments the RF beam generators may be tuned to emit beams at different frequencies, and personnel program the RF beam generators to emit RF beams at different frequencies. In some further embodiments different RF beam generators emit beams at different frequencies at different times. In some further embodiments different RF beam generators all emit at the same frequency. In some other further embodiments different RF beam generators emit at varying frequencies at the same time.

In some embodiments personnel program the machine to fire beams for particular durations.

In some embodiments personnel program the machine to fire beams at particular intensities.

In some embodiments personnel program the machine to fire some RF beam generators and not others, thus allowing finer control over the intensity of energy at the focus. This also allows personnel to optimize where the RF beams traverse the patient's non-target tissue for various reasons, e.g. to avoid tissue that is impervious to the RF beams.

In some embodiments personnel program different RF beams at different frequencies.

In some embodiments personnel program different RF beams to fire at different intensities.

In some embodiments personnel program different RF beams to fire at different times.

In some embodiments personnel program different RF beams to fire for different durations.

In some embodiments the machine comprises other mechanisms to determine features inside living tissue. Such features include but are not limited to tumors, bone, muscle, connective tissue, fatty deposits, and organs. Such mechanisms include but are not limited to X-rays, NMR and ultrasound. In some embodiments the machine contains mechanisms to detect when, as the living tissue moves, the focus coincides with the intended target. In some embodiments the machine works in tandem with other machines which determine when this happens. In some further embodiments the machine is programmed to energize its RF beams at precisely those times when the focus coincides with the intended target.

In some embodiments the machine uses a thermometer to measure temperature at the focus and in other tissue. In some embodiments the machine uses some non-invasive means to measure such temperature.

In some embodiments the machine holds no such apparatus to measure temperature.

In some embodiments the machine incorporates other courses of treatment including but not limited to ultrasound, focused ultrasound heating, and gamma radiation.

### Combination Embodiments

In some embodiments the machine works with some other mechanism to heat the target and the tissue surrounding it, elevating the temperature of all this tissue to below the threshold for thermal damage. Techniques for this general heating include but are not limited to infrared radiation, ultrasound, conduction and convection. Then the machine heats the target with multiple directed RF beams enough to cause the target tissue to ablate. The advantage of this technique over using RF beams alone is that the amount of temperature increase due to RF alone does not need to be as much.

In some embodiments the machine works with other machines that provide other courses of treatment including but not limited to ultrasound, focused ultrasound heating, gamma radiation therapy, chemotherapy, and nanoparticle therapy.

In some embodiments to treat a tumor a patient receives a chemotherapy treatment drug, and this drug travels all or part of the patient's body. These travel all over the body, including into the target. Then the RF generators emit RF beams which focus on the target, where those RF beams are tuned to a frequency which excites the given chemotherapy trug only at the target.

In some embodiments nanoparticles, such as but not limited to gold nanoshells and nanorods, superparamagnetic iron oxide particles and carbon nanotubes, are introduced into the patient's body. These travel all over the body, including into the target. Then the RF generators emit RF beams which focus on the target, where those RF beams are tuned to a frequency which excites the given nanoparticles only at the target.

### Further Embodiments

We show these alternatives to be exemplary and in no way limiting. The embodiments of the invention are various and numerous, without departing from its spirit or sacrificing its advantages.

### Advantages

This technique has advantages over the various existing techniques heat-treating various forms of target.

Surgical removal indeed eliminates tumors, cysts, and ulcers. And surgical techniques may stop internal bleeding or other wounds. But surgery itself affects the body of the patient. Some tumors are inoperable. Some internal bleeding sites are untreatable. And even when a tumor or other site is operable surgery is traumatic; it causes systemic shock, and also results in scar tissue. It may lead to the introduction into the patient's body of undesired cells, microbes or toxins.

Radiation treatment is an effective way to treat tumors, but alas, it is difficult to direct radiation to strike a tumor alone, without also affecting a significant amount of the surrounding tissue. One conventional way to use RF to ablate tissue is to penetrate the tissue to ablate with a needle emitter which generates RF from its tip. But this technique has the disadvantage that it is by definition surgically invasive.

Gamma knife radiation treatment seems very effective at treating those types of tumors that respond well to radiation. But it is very expensive. The inventions presented here are much more economical. Chemotherapy is also effective against tumors, but again by its very nature is toxic to the body. Chemotherapy effectively uses poisons to kill tumors, which are more susceptible to such poisons than non-cancerous tissue. But even so chemotherapy drugs are poisonous to the rest of the body as well, and weakens the body.

A single metamaterial lens generating a directed beam where all the energy converges at the focus, could in theory achieve a result somewhat similar to that of this invention. However, a single such beam would damage not just the tissue intended for ablation, but also the tissues surrounding it, since the RF beam would get ever stronger as it neared the focus, and then weaken gradually as it passed the focus.

Focused ultrasound techniques may provide potential courses of localized thermal treatment that may compete with the machine and courses of treatment disclosed here. However, it is likely that ultrasound at various frequencies, and RF at various frequencies, provide potentially different levels of effectiveness. This arises due to how ultrasound at different frequencies, and RF at different frequencies:
penetrate tissue
dissipate into heat energy in tissue
disperse, thus losing concentration, in tissue
reflect and/or refract in tissue particularly at boundaries such as between bone and connective tissue, or muscle and fatty tissue

So it is likely that RF and ultrasound techniques will complement each other as potential courses of thermal ablation treatment.

The invention presented here offers the hope of being able to target very specific target tissue and nothing else, without invasive surgery and the complications thereof.

### CONCLUSIONS, RAMIFICATIONS AND SCOPE

The hope is the invention presented here will augment the arsenal of techniques and machines to be used against cancer, and using these inventions either by themselves, or in conjunction with other techniques such as radiation therapy, chemotherapy and surgery, will enhance the chance of surviving cancer.

Also the hope is the inventions presented here will enable easier, cheaper and more effective treatment of various other ailments, from ulcers to removal of undesirable fatty deposits, than surgery might.

We claim:

1. A machine comprising multiple RF beam generators where all said RF beam generators are spatially oriented for their beams all to pass through a region of space, called the focus, which is completely contained within some sphere of some diameter greater than or equal to 0.001 mm, and less than or equal to 10 cm.

2. A method of the machine of claim 1 where:
a) a patient with a piece of tissue to be treated by heat, called a target, is positioned such that the focus of said RF beam generators in said machine all pass through said target;
b) the machine energizes said RF beam generators, which produce RF beams which converge at said target;
for the purpose of providing heat treatment to said target without affecting other tissue in said patient.

3. The method of claim 2 where nanoparticles are introduced into the patient's body and some of these nanoparticles are activated by the machine producing heat; thereby treating the target more effectively than heat alone would.

4. The method of claim 2 where a plurality of chemotherapy drugs is introduced into the patient's body and some of these nanoparticles are activated by the machine producing heat; thereby treating the target more effectively than heat alone would.

5. The machine of claim 1 where said machine comprises a plurality of programmable automata, where said programmable automata control said machine.

6. The machine of claim 5 where each said RF beam generator is attached to the framework of said machine in a manner that its angular offsets from said frame is adapted to be adjusted by devices or by hand labor.

7. The machine of claim 6 where each said RF beam generator is attached to a plurality of servo-motors which control the angular offsets of said RF beam generator from the framework of said machine.

8. The machine of claim 7 which comprises a sensor that senses RF beams.

9. The machine of claim 8 where said sensor is adapted to be placed in the path of the RF beams said RF beam generators generate.

10. A method of the machine of claim 9 where:
a) said sensor is locked to a desired location;
b) for each said RF beam generator:
<i> said machine uses said servo-motors to sweep said RF beam generator through all possible angles until sensor indicates it is specifically targeted by said RF beam;
<ii> said machine uses said servo-motors to lock said RF beam generator to said angular position;
thereby calibrating all said RF beam generators in said machine to be optimally oriented to strike the targeted focus.

11. The machine of claim 1 further comprising additional mechanisms to heat tissue drawn from the set of conduction, convection, infrared radiation, and ultrasound.

12. A method of the machine of claim 11 where:
a) a patient with a piece of tissue to be treated by heat, called a target, is positioned such that the focus of said RF beam generators in said machine all pass through said target;
b) said additional heating mechanisms elevate the temperature of the target and the tissue surrounding the target to a level insufficient to cause tissue damage;
b) the machine energizes said RF beam generators, which produce RF beams which converge at said target;
for the purpose of providing heat treatment to said target without heating non-target tissue sufficiently to damage it.

13. The machine of claim 1 which receives feedback from sensors which determine the exact times at which the desired part of said patient's body are at said focus.

14. A method of the machine of claim 13 where said machine energizes its RF generators only when said desired part of said patient's body are at said focus.
